(19) United States
(12) Patent Application Publication
(10) Pub. No.: US 2008/0213252 A1
(43) Pub. Date: Sep. 4, 2008

(54) METHODS OF TREATING ITCH

(76) Inventors: Ethan LERNER, Chestnut Hill, MA (US); Vemuri B. REDDY, Ashland, MA (US)

Correspondence Address: DAVID S. RESNICK, NIXON PEABODY LLP, 100 SUMMER STREET, BOSTON, MA, 02110-2131, US

(21) Appl. No.: 11/968,800

(22) Filed: Jan. 3, 2008

Related U.S. Application Data
(60) Provisional application No. 60/878,302, filed on Jan. 3, 2007.

(51) Int. Cl. A61K 39/395; A61K 31/35; A61K 31/40; A61K 31/404; A61P 17/00

(52) U.S. Cl. 424/130.1; 424/141.1; 514/460; 514/423; 514/419

(57) ABSTRACT

The invention provides methods for preventing and/or treating itch in a subject in need thereof by administration of a protease-activated G-protein coupled Protease Activated Receptor 4 (PAR4) antagonist. The PAR4 antagonist can be combined with a PAR2 antagonist.

### CROSS REFERENCE TO RELATED APPLICATIONS

This application claims benefit of the U.S. Provisional application No. 60/878,302 filed Jan. 3, 2007, the contents of which are incorporated herein by reference in its entirety.

### GOVERNMENT SUPPORT

This invention was made with Government support under Grant No.: P01 NS 047300 and UL1 RR024139-01 awarded by the National Institutes of Health. The Government has certain rights in the invention.

### FIELD OF THE INVENTION

The invention relates to methods of preventing and/or treating itch. In particular, the invention relates to blocking PAR4 receptor protease-activated events using PAR4 antagonists.

### BACKGROUND OF INVENTION

Itch, or pruritus, is the unpleasant sensation that leads to a desire to scratch and is a common and distressing symptom in a variety of conditions and diseases. Itch typically occurs in peripheral diseases such as allergic conjunctivitis, allergic rhinitis, hemorrhoids, and dermatoses of fungal, allergic and non-allergic origin. Itching can also be a major symptom of many systemic diseases such as, Hodgkin's disease, chronic renal failure, polycythemia vera, hyperthyroidism and cholestasis. In addition, senile itch without an obvious cause, except perhaps xerosis, occurs in more than half of the population aged 70 years. In all cases chronic severe generalized itch can be disabling.

The intracutaneous injection of histamine or proteases (trypsin, tryptase, papain) elicit itch, and this has been used as an experimental model for itch studies. It was, therefore, postulated that these agents are involved as mediators in various itching conditions. However it has become apparent that central transmission of the itch sensation involves more than histamine or proteases. For example, cholestasis is characterized by generalized itch, which is not responsive to H1-antihistamines, indicating that histamine is probably not the major mediator involved. Approximately 25% of patients with uremia (chronic renal failure) suffer from severe itch unresponsive to H1-antihistamines or dialysis. However since histamine was believed to be the primary mediator of the itch sensation, conventional itch therapy involves H1-antihistamines as a first-line medication although antihistamines have no general anti-pruritic effect, and in many instances they are either ineffective or only partially effective.

Itching can be elicited by chemical, electrical, mechanical and thermal stimulation. So far no morphological structure has been identified as a specific receptor for the itch sensation, but it is assumed that itch receptors are linked to the free nerve endings of C-fibers close to the dermo-epidermal junction. The impulses set up in the thin, non-myelinated, slowly conducting C-fibers enter the spinal cord via the dorsal horn, then ascend in the contralateral spinothalmic tract, pass via the thalamus and end in the somatosensory cortex of the post-central gyrus. Itching and pain are related phenomena, and it was previously believed that itching was equal to sub-threshold pain, i.e. with increased activity in the C-fibers the perceived sensation changed from itching to pain. Although itch was once thought to be a subliminal form of pain (intensity theory), current evidence points to separate sensory neuronal systems mediating the two modalities. First, pain and itch are dissociable. Pain and itch evoke different motor responses, scratching for itch and withdrawal for pain. Second, based on clinical observations, systemically-administered opioids have a dichotomous effect on these two sensory modalities. μ-opioid receptor agonists reduce pain but can cause itch. Furthermore, antagonizing the central μ-opioid receptors, for example with naloxone or naltrexone, suppresses pruritus and at the same time may lower the pain threshold.

There are several topical and systemic agents that suppress itching in selected clinical settings. Unfortunately, no universally effective anti-pruritic drug exists. Attempts to develop specific drugs have not been successful, partly because the transmission pathways for itch are not well understood. Therefore, there is an urgent need for new approaches for managing itch.

### SUMMARY OF THE INVENTION

The inventors has discovered that the G-protein coupled protease activated receptor 4 (PAR4) is a key effector of the itch sensation caused by a cysteine protease that was isolated and identified from the plant Mucuna pruriens, also known as cowhage, referred to as mucunain. It has also been discovered that mucunain cleaves the PAR4 and PAR2, which then induce influx of calcium into the affected cells, thus eliciting a signal transduction event towards the sensation of itch.

Accordingly, in one embodiment, the present invention provides a method of preventing and/or treating itch in a subject in need thereof by administering a therapeutically effective amount of a PAR4 antagonist.

In one embodiment, the invention disclosed herein is suitable for the prevention and/or treatment of itch that is associated with a disease or disorder selected from eczema, atopic eczematous dermatitis, seborrheic dermatitis, atopic dermatitis, contact dermatitis, irritant dermatitis, xerosis (dry skin), psoriasis, fungal infections including athlete's foot, yeast infections including diaper rash and vaginal itch, parasitic infections, parasitic infestations including scabies and lice, lichen planus, lichen simplex, lichen simplex chronicus, lichen sclerosis, itch secondary to medications, senile itch, uremia, idiopathic itch, itch associated with liver cirrhosis, itch associated with inflammation, itch associated with allergies, itch associated with cancer, itch associated with kidney disease, itch associated with haemodialysis, burns, scalds, sunburn, wound healing, insect bites, urticaria, sweat gland abnormalities, bullous pemphigoid, photodematoses, skin blisters, adult acne, chicken pox, and dermatitis herpetiformis.

In one embodiment, the invention disclosed herein utilizes a PAR 4 antagonist, for example, tcY-NH(₂); pepducin P4 pal-10; pepducin P4 pal-15; Tc-APGKF-NH(₂) (SEQ. ID. No. 26); polyclonal anti-PAR4 antibody; monoclonal anti-PAR4 antibody, YD-3; Statins: atorvastatin, pravastatin, fluvastatin, cerivastatin, lovastatin, simvastatin, rosuvastatin, pitavastatin, and metabolite thereof; and ethanol.

In one embodiment, the PAR4 antagonist is applied topically to the site afflicted with itch. In another embodiment, the PAR4 antagonist is administered systemically. In one embodiment, the PAR4 antagonist is administered in the form of a pharmaceutical composition comprising a therapeutically effective amount of said PAR4 antagonist as the active ingredient, in admixture with a pharmaceutical carrier. For topical application, the PAR4 antagonist pharmaceutical composition can be formulated in a form suitable form for topical application such as a skin patch.

In one embodiment, the PAR4 antagonist is administered in conjunction with PAR2 antagonist, for example, the synthetic peptide FSLLRY-NH₂ (SEQ. ID. No. 31), the small molecule ENMD-1068: N(1)-3-methylbutyryl-N(4)-6-aminohexanoyl-piperazine, PAR2 monoclonal antibody, SAM-11 and P2pal-21 (Covic, J., et. al., 2002, PNAS, 99:643-648).

In one embodiment, reconstituted spicules from the pods of the tropical plant M. pruriens can be used as a carrier of the PAR4 antagonist pharmaceutical composition.

In one embodiment, the invention provides a method of preventing and/or treating itch in a subject in need thereof by administering a therapeutically effective amount of the cysteine protease inhibitor, E-64, to the subject.

### BRIEF DESCRIPTION OF THE INVENTION

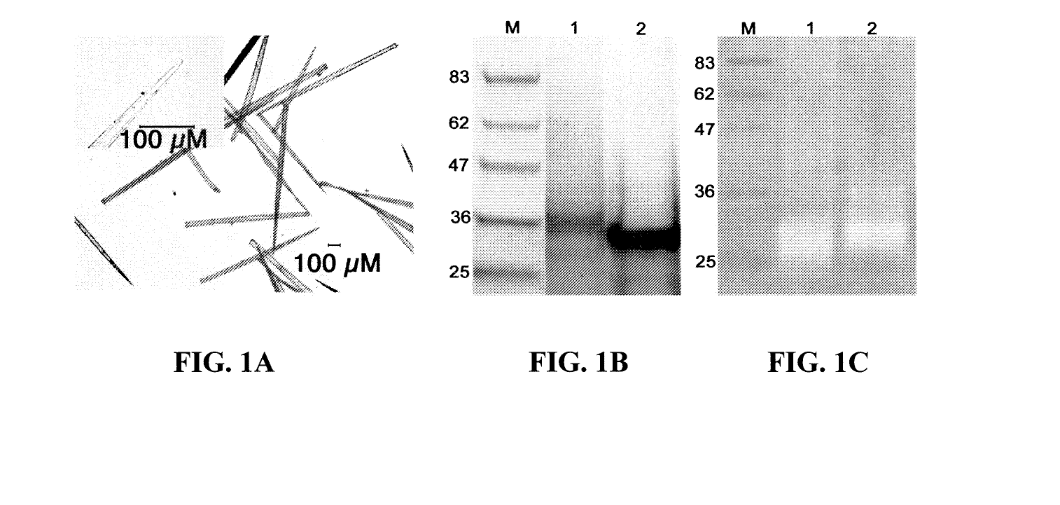

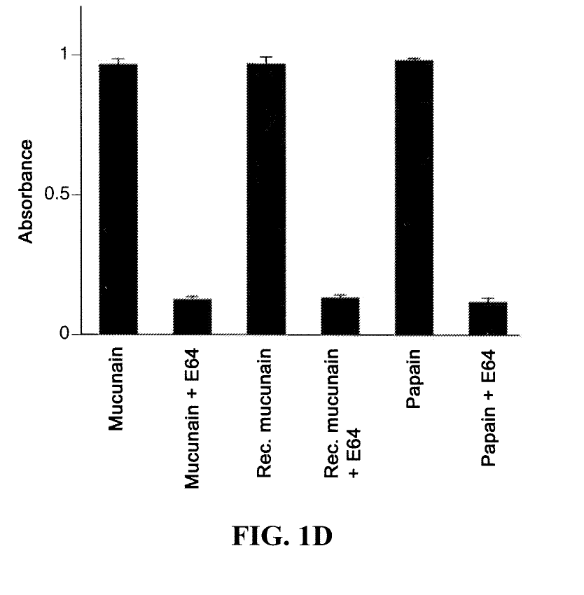

FIG. 1A. Photomicrograph of native spicules. Each spicule is 2-3 mm in length and typically one or a few microns in diameter at the tip.

FIG. 1B. SDS-PAGE gel electrophoresis of M. pruriens extract (lane 1) and recombinant mucunain (lane 2). Molecular weight markers (kDa) are shown on the left.

FIG. 1C. Casein zymogram gels showing the proteolytic activity of the M. pruriens extract (lane 1) and of the recombinant material (lane 2). Molecular weight markers (kDa) are shown on the left.

FIG. 1D. Protease cleavage activity of Mucunain. Experiments were performed in triplicate and the error bars represent standard deviation.

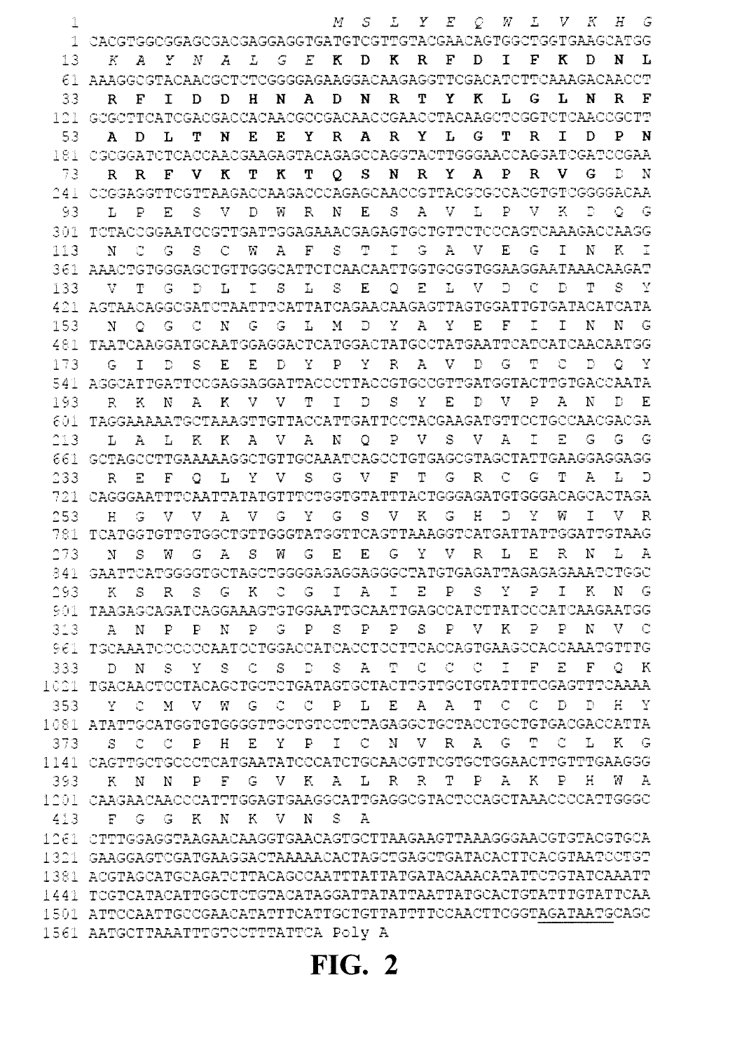

FIG. 2. Nucleotide and predicted amino acid sequence of mucunain (SEQ. ID. No. 32 and 33). The 20-residue signal peptide is in italics. The 70-residue pro-region of the zymogen is in bold. The mature protein is predicted to contain 332 amino acids. The predicted polyadenylation site is underlined.

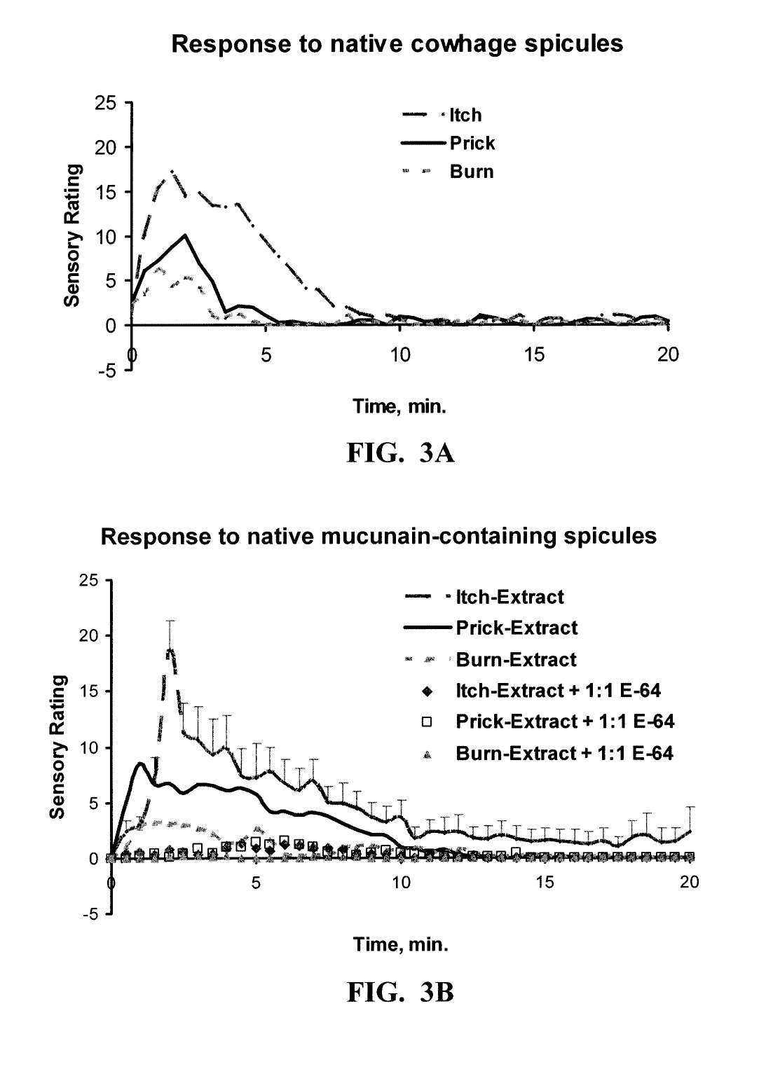

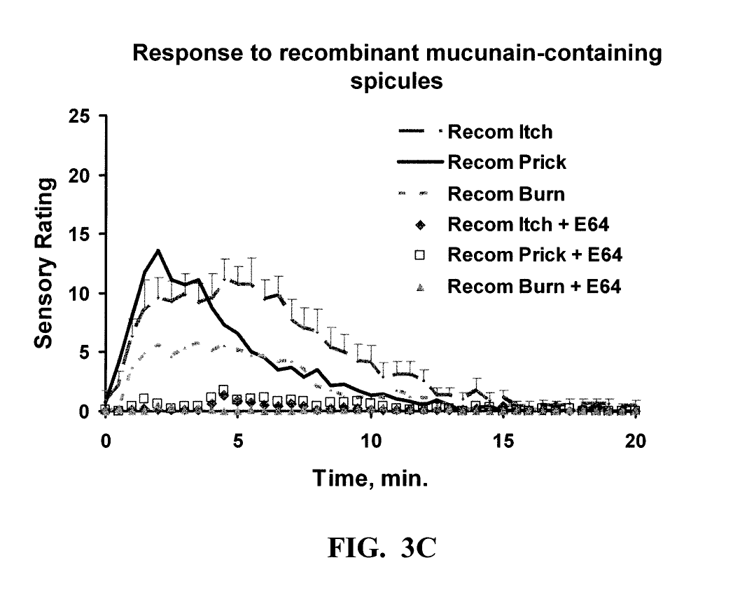

FIG. 3A. Magnitude and time course of sensations evoked by native cowhage spicules.

FIG. 3B. Magnitude and time course of sensations evoked by spicules containing only native mucunain.

FIG. 3C. Magnitude and time course of sensations evoked by spicules containing only recombinant mucunain.

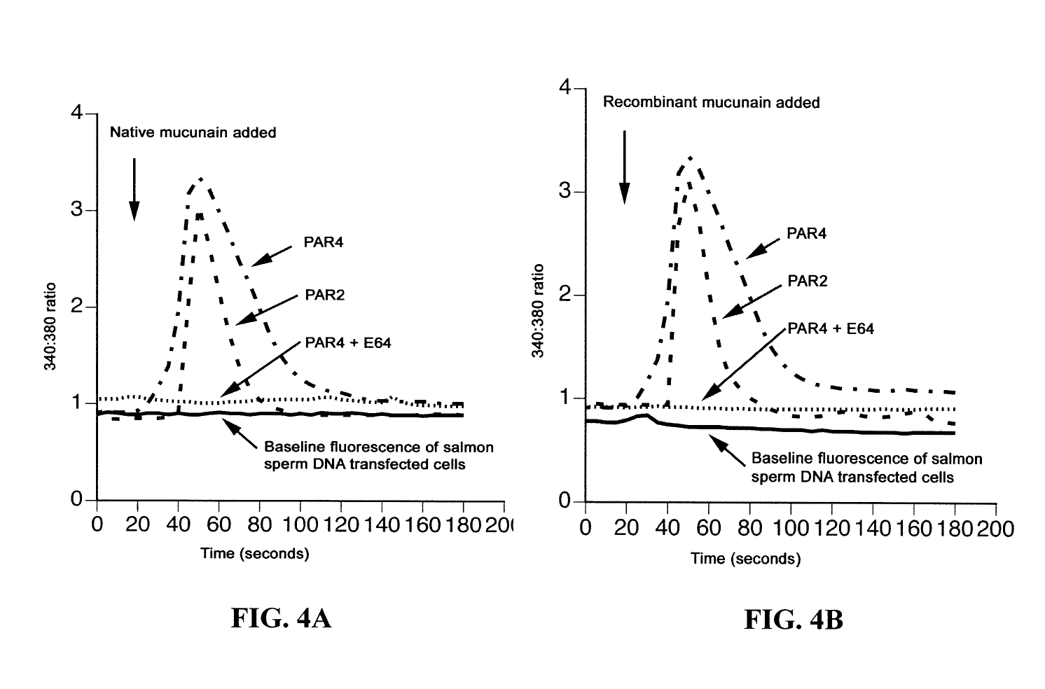

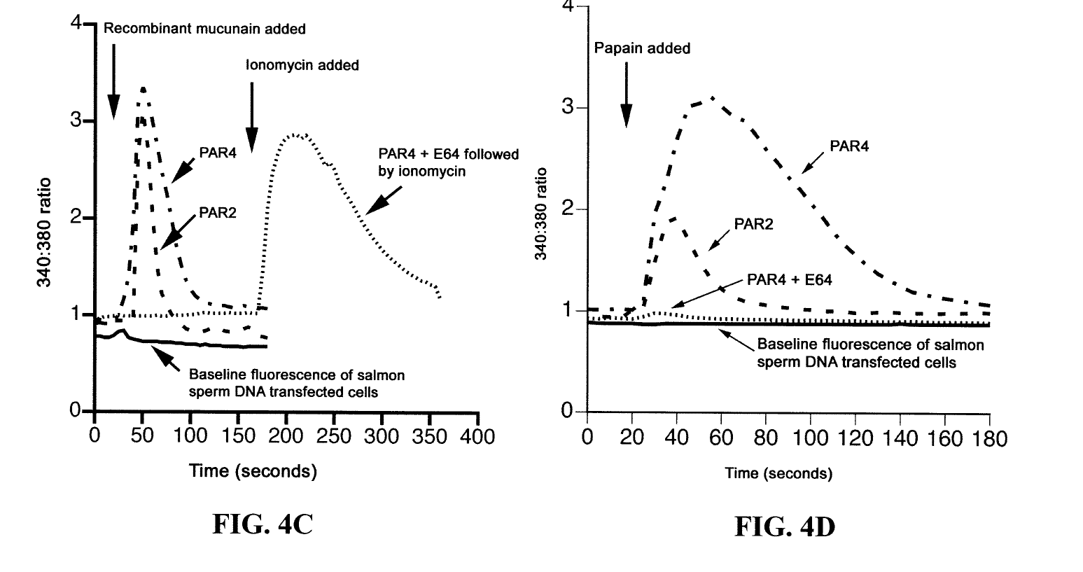

FIG. 4A. Native mucunain induced on calcium release in HeLa cells transfected with PAR receptors. Data from single cell imaging.

FIG. 4B. Recombinant mucunain induced on calcium release in HeLa cells transfected with PAR receptors. Data from single cell imaging.

FIG. 4C. Ionomycin induced on calcium release in HeLa cells transfected with PAR receptors treated with cysteine protease inhibitor E64. Data from single cell imaging.

FIG. 4D. Papain induced on calcium release in HeLa cells transfected with PAR receptors. Data from single cell imaging.

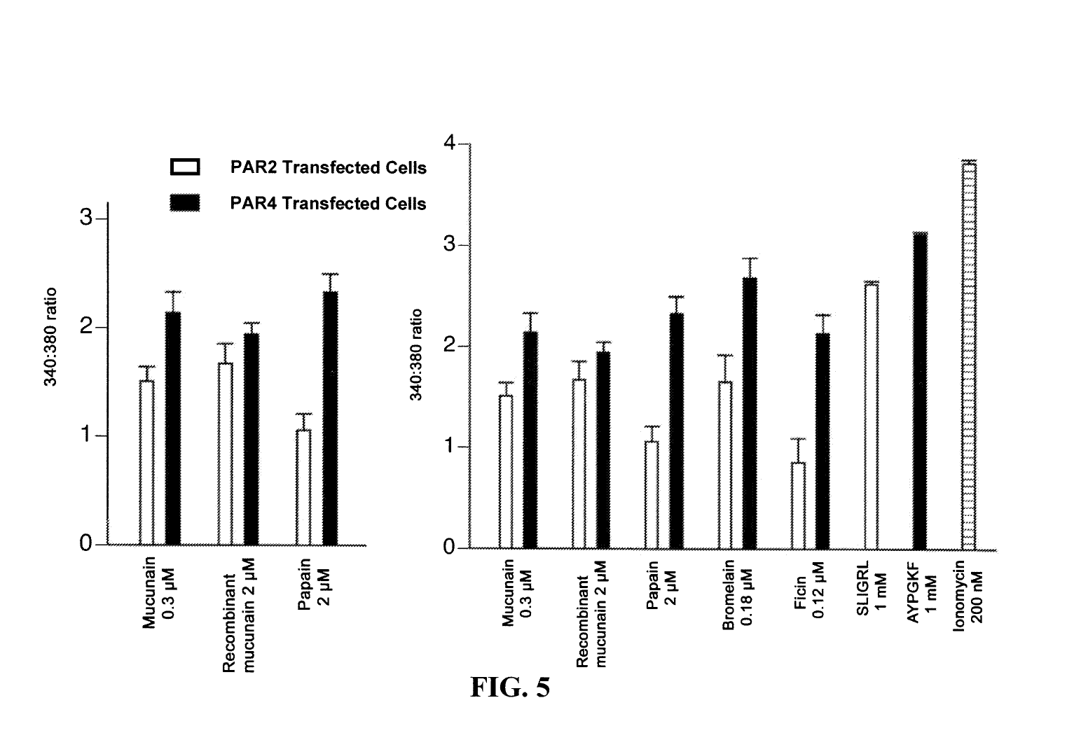

FIG. 5. Peak 340:380 ratiometric calcium imaging response to the indicated protease of cells transfected with cDNAs encoding PAR2 (clear bars) and PAR4 (solid bars).

### DETAILED DESCRIPTION OF INVENTION

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Definitions of Terms

As used herein, the term “itch”, technically known as pruritus refers to the sensation that elicits a reflex response to scratch. Itch can be a symptom of a disease, disorder or infection, or itch can arise spontaneously, without an underlying or identifiable physiological cause, known as idiopathic pruritus.

As used herein, the term “antagonist” in “a PAR2 antagonist” or “PAR4 antagonist” refers to any organic or inorganic molecule that opposes the naturally occurring signaling events elicited by protease-activated PAR protein respectively. For example, an antibody that blocks the interaction of a protease and PAR protein, and there by preventing the cleavage-activation of the PAR signaling pathway.

As used herein, the term “treatment” refers to all aspects of control of itching including prophylaxis and therapy. Control of itch of include reducing, alleviating, relieving and numbing the sensation of itch. Control of itch also include reducing the desire to scratch.

The term “therapeutically effective amount” and grammatical variations thereof, as used herein refer to sufficient quantities of an active compound that can produce the desired therapeutic effect when administered to a mammal afflicted with pruritus. The term “therapeutic effect” is used herein in a broad sense and includes prophylactic effects.

As used herein, the term “antibody” refers to immunoglobulin molecules and immunologically active portions of immunoglobulin molecules, i.e., molecules that contain an antigen binding site that immunospecifically binds an antigen. The terms also refers to antibodies comprised of two immunoglobulin heavy chains and two immunoglobulin light chains as well as a variety of forms besides antibodies; including, for example, Fv, Fab, and F(ab)′2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference.).

In accordance with the present invention, the itch-inducing component of spicules from the plant M. pruriens, also known as cowhage, has been discovered. Spicules from M. pruriens constitute a true ‘itching powder’. Their small size, 0.1×2 mm, allows the tip to penetrate the epidermis and cause intense itching but no other changes in the skin, such as a wheal and flare or urticaria. The newly discovered novel pruritogen is a cysteine protease and its corresponding itch receptor is the G-protein coupled protease activated receptor 4 (PAR4). The cysteine protease is named mucunain. Serine proteases, such as trypsin or tryptase from mast cells, are the known protease-type itch mediators, stimulating the another protease activated receptor, PAR2, present in skin. The inventor has discovered that stimulation of the PAR4 receptor by two known PAR4 small peptide agonists causes itching in human volunteers. The activation of PAR4, most likely on nerves in the skin, turns on a signaling cascade, the result of which is manifest as the desire to scratch. Accordingly, inhibiting PAR4 is useful in the treatment or prevention of itching. Additionally, in another embodiment, the spicules of M. pruriens can be used as a percutaneous delivery system.

The protease-activated receptor 4 (PAR4) is a member of protease activated receptor (PAR) family of receptors that are activated by protein cleavage. There are four known PAR protein, PAR1, PAR2, PAR3, and PAR4, and they are activated by trypsin, tryptase, and thrombin, all serine proteases. Thrombin acts on PAR1, PAR3, and PAR4, while trypsin and tryptase act on PAR2. The PAR family of receptors is a subset of G-protein coupled receptors (GPCR) which are sensors that are activated by a variety of triggers, including light, taste, olfaction, pain and temperature.

In one embodiment, the present invention provides a method of preventing and/or treating itch in a subject in need thereof by administering a therapeutically effective amount of a PAR4 antagonist. The PAR 4 antagonist is selected from the antagonist set forth in Table 1. These include but are not limited to tcY-NH(2); pepducin P4 pal-10; pepducin P4 pal-15; Tc-APGKF-NH(₂) (SEQ. ID. No. 26); polyclonal anti-PAR4 antibody; monoclonal anti-PAR4 antibody, YD-3; Statins: atorvastatin, pravastatin, fluvastatin, cerivastatin, lovastatin, simvastatin, rosuvastatin, pitavastatin, and metabolite thereof; and ethanol. The anti-PAR4 antibodies that block the protease-dependent activation of PAR4 are preferred, for example, an antibody that binds to or binds close to the protease cleavage site so that steric hindrance by the bulky antibody prevents the protease from cleaving the PAR4 protein. For example, antibodies raised against the C-terminal 50 amino acids portion of the PAR4 protein such as anti-PAR4 (C-19) (sc-1249, Santa Cruz Biotechnology, Inc.). Encompassed in the invention are conjugates of antibodies, e.g. Fv, Fab, and F(ab)′2, bifunctional hybrid antibodies and single chain antibodies that are known to one skilled it the art.

In one embodiment, the method of preventing and/or treating itch in a subject in need thereof involves administering a therapeutically effective amount of a PAR4 antagonist in conjunction with a therapeutically effective amount of a PAR2 antagonist. The PAR2 pathway has been shown to be involved in itch in the human skin (Steinhoff, M., et. al., 2003, J. Neurosci. 23: 6176-80). Examples of PAR2 antagonists include but are not limited to the synthetic peptide FSLLRY-NH₂ (SEQ. ID. No. 31) (PAR-3888-PI) (Peptides International) (Al-Al-Ani, B. et. al., 2002, J. Pharmacol. Exp. Ther. 300:702-8), the small molecule ENMD-1068: N(1)-3-methylbutyryl-N(4)-6-aminohexanoyl-piperazine (Kelso, E B. et. al., Arthritis Rheum. 2007 Feb. 27; 56 (3):765-771), PAR2 monoclonal antibody, SAM-11 (sc-13504, Santa Cruz BioTechnology, Inc.), and P2pal-21 (Covic L., et. al., 2002, supra).

The inventor has demonstrated that the cysteine protease synthetic peptide inhibitor, E-64, was effective in preventing itch brought about by mucunain. E-64 [1-[N-[(L-3-trans-carboxyoxirane-2-carbonyl)-L-leucyl]amino]-4-guanidinobutane] is an irreversible inhibitor of many cysteine proteases. Accordingly, in one embodiment, the invention provides a method of preventing and/or treating itch in a subject in need thereof by administering a therapeutically effective amount of the cysteine protease inhibitor, E-64, to the subject. In another embodiment, the therapeutically effective amount of E-64 is administered in conjunction with a PAR4 antagonist. In yet another embodiment, the therapeutically effective amount of E-64 is administered in conjunction with a PAR2 antagonist.

In another embodiment, the methods disclosed herein can be used in conjunction with other known anti-itch therapies such as menthol and phenol, calamine, topical antihistamines, local anesthetics, capsaicin, strontium nitrate, H1-receptor antagonists, H2-receptor antagonists, doxepin, ondansetron, paroxetine, mirtazapine, opioid antagonists, dry skin: emollient cream, cholestasis: colestyramine, rifampicin, opioid antagonists, androgens, for uremia: dialysis, UVB phototherapy and for paraneoplasia: paroxetin.

In another embodiment, the methods disclosed herein can be used in conjunction with therapies for eczema, atopic eczematous dermatitis, seborrheic dermatitis, atopic dermatitis, contact dermatitis, irritant dermatitis, xerosis (dry skin), psoriasis, fungal infections including athlete's foot, yeast infections including diaper rash and vaginal itch, parasitic infections, parasitic infestations including scabies and lice, lichen planus, lichen simplex, lichen simplex chronicus, lichen sclerosis, itch secondary to medications, senile itch, uremia, idiopathic itch, itch associated with liver cirrhosis, itch associated with inflammation, itch associated with allergies, itch associated with cancer, itch associated with kidney disease, itch associated with haemodialysis, burns, scalds, sunburn, wound healing, insect bites, urticaria, sweat gland abnormalities, bullous pemphigoid, photodematoses, skin blisters, adult acne, chicken pox, and dermatitis herpetiformis.

In accordance with the present invention, the PAR4 antagonists are preferably applied topically to the site afflicted with itch in therapeutically effective amount in admixture with pharmaceutical carriers, in the form of topical pharmaceutical compositions. Such compositions include solutions, suspensions, lotions, gels, creams, ointments, emulsions, skin patches, etc. All of these dosage forms, along with methods for their preparation, are well known in the pharmaceutical and cosmetic art: Harry's Cosmeticology (Chemical Publishing, 7th ed. 1982); Remington's Pharmaceutical Sciences (Mack Publishing Co., 18th ed. 1990). Typically, such topical formulations contain the active ingredient in a concentration range of 0.001 to 10 mg/ml, in admixture with suitable vehicles. Other desirable ingredients for use in such anti-pruritic preparations include preservatives, co-solvents, viscosity building agents, carriers, etc. The carrier itself or a component dissolved in the carrier may have palliative or therapeutic properties of its own, including moisturizing, cleansing, or anti-inflammatory/anti-itching properties. The PAR4 antagonists can be combined with a therapeutically effective amounts of anti-inflammation agents such as corticosteroids, fungicides, antibiotics, moisturizers or anti-itching compounds.

Penetration enhancers may, for example, be surface active agents; certain organic solvents, such as di-methylsulfoxide and other sulfoxides, dimethyl-acetamide and pyrrolidone; certain amides of heterocyclic amines, glycols (e.g. propylene glycol); propylene carbonate; oleic acid; alkyl amines and derivatives; various cationic, anionic, nonionic, and amphoteric surface active agents; and the like.

Topical administration of a pharmacologically effective amount may utilize transdermal delivery systems well known in the art.

In addition to topical therapy, the PAR4 antagonists are also administered systemically, such as oral, parenteral, nasal inhalation, and intrarectal are also contemplated. For these uses, additional conventional pharmaceutical preparations such as tablets, granules, powders, capsules, and sprays may be preferentially required. In such formulations further conventional additives such as binding-agents, wetting agents, propellants, lubricants, and stabilizers may also be required.

Systemic administration preferably comprises ingestion of any solid or solution carriers containing a pharmacologically effective amount of one or more of the PAR4 antagonists. Such solid or solution carriers may comprise pills, hard tablets, soft tablets, gums or ordinary liquids. Additionally, systemic administration of a pharmacologically effective amount may comprise invasive methodologies including intravenous, subcutaneous, intramuscular or intralesional injection of a suitable carrier, such as saline, containing a pharmacologically effective amount of one or more of the PAR4 antagonists.

The route of administration, dosage form, and the effective amount vary according to the potency of the selected PAR4 antagonist, its physicochemical characteristics, and according to the location of itch sensations. The selection of proper dosage is well within the skill of an ordinary skilled physician. Topical formulations are usually administered up to four-times a day.

The use of PAR4 antagonists as anti-pruritics is advantageous in that they relieve itching by a mechanism independent of histaminergic compounds. Thus, they may be effective in itching diseases which are refractory to antihistamine therapy and may be combined with H1-antihistamines to provide superior therapy via additive or synergistic interaction.

The itch may be associated with a disease or disorder related from the group consisting of eczema, atopic eczematous dermatitis, seborrheic dermatitis, atopic dermatitis, lichen planus, senile itch, uremia, idiopathic itch, itch associated with liver cirrhosis, itch associated with inflammation, itch associated with allergies, itch associated with cancer, itch associated with haemodialysis, burns, scalds, sunburn, insect bites, urticaria, sweat gland abnormalities, bullous pemphigoid, photodematoses, skin blisters, adult acne, chicken pox, and dermatitis herpetiformis.

This invention also provides a novel and less invasive delivery method for cutaneous administration of drugs. Inactivated spicules from the pods of the tropical plant M. pruriens acts as minute hypodermic syringes. The inactivated spicules are uploaded with the drug by soaking the spicules in a solution containing the drug. The spicules may be inactivated by use of high heat and pressure, for example autoclave. In one embodiment, the drug is soluble and active in solvent. The reconstituted spicules is then formulated for topically application.

This invention is further illustrated by the following examples which should not be construed as limiting. The contents of all references, patents, and patent applications cited throughout this application, as well as the figures, table, and sequence listing are incorporated herein by reference.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

### EXAMPLE

### Materials and Methods

M. pruriens or cowhage grows wild in numerous tropical areas. Materials were obtained, including spicules, pods, leaves and stems from several geographic locations including Costa Rica, Venezuela, Cameroon and India. Regardless of source, the cowhage, at least subjectively in our hands, has similar pruritus-inducing activity in human skin. A variety of extraction, separation and mass spectrometric techniques were employed in the effort to isolate and identify the active component. Again, the results were similar regardless of geographic origin of the material. Both small molecules and protein peaks and bands were observed. One of the small molecules was found to be folic acid, a common component of plants. Only the data relevant to the active cysteine protease is presented.

Chemicals—Crystallized papain was obtained from Sigma.

Protein extraction from spicules—Dry pods of M. pruriens were obtained from Costa Rica. The spicules were collected by shaking the pods in a closed bag in a chemical hood to decrease the chance of spreading itch-inducing spicules in the laboratory. Ten grams of spicules were stirred at 40° C. for 2 hours in 300 ml of a buffer containing 0.1 M NaCl and 0.01 M L-cysteine, pH 5.7. This crude extract was passed through cheesecloth and then a 0.4 μM filter. The extract was size-selected using centrifugal concentrators that had a molecular weight cut-off of 5 kDa. It was further purified using a P-60 column and fractions were run on SDS-PAGE gels. The fraction showing a single prominent band at 32 kDa was subjected to further analysis. This material is referred to as native mucunain and quantified using a non-interfering protein assay (Pierce). A photomicrograph of native spicules are shown in FIG. 1A.

Protein sequencing—The protein band running at 36 kDa was subjected to amino acid sequence analysis by tandem mass spectrometry on a Finnigan LCQ quadrupole ion trap mass spectrometer. The protein was also subjected to NH₂-terminal sequence analysis by Edman degradation.

Preparation of total RNA, poly-A RNA and ds cDNA—RNA was extracted from leaves and stems as it was found that spicules did not contain workable quantities of RNA. Leaves and stems of M. pruriens were ground to a fine powder in liquid nitrogen and total RNA was extracted using the Qiagen RNAeasy protocol. Poly-A RNA was isolated by Qiagen's Oligotex procedure. The poly-A RNA was converted into ds cDNA using a cDNA preparation kit from Stratagene.

Cloning of M. pruriens cysteine protease cDNA by PCR—One μg of ds cDNA was used as a template in a 50 μl PCR volume using a forward primer, TGG GGC GCC AGC TGG GGY TTC GAG GGY TAY (SEQ. ID. No. 2) based on the internal peptide, NSW GAS WGF EGY VR (SEQ. ID. No. 3), and a reverse primer, dT28. The reaction containing Pfu DNA polymerase from Stratagene was heated at 95° C. for 5 minutes followed by 30 cycles of denaturation at 95° C. for 30 seconds, annealing at 55° C. for 1 minute, extension at 68° C. for 2 minutes and a final extension at 68° C. for 10 minutes. PCR products greater than 100 bp in length were cloned into a Zero-blunt TOPO PCR vector (Invitrogen) and sequenced. A reverse primer, AGG ACA AAT TTA AGC ATT GCT GCA TTA TCT ACC (SEQ. ID. No. 4) (based on the C-terminal sequence near the poly-A tail) and a forward primer, GAT AAC TTG CCG GAA TCT GTT GAT TGG AG (SEQ. ID. No. 5) (based on the NH₂ terminal peptide, DNL PES VDW RNE GAV LPC KS, (SEQ. ID. No. 6)) were used to isolate the entire mature protein coding sequence of 1300 bp. The entire cDNA of 1600 bp was later isolated using a forward primer, CAC GTG GCG GAG CGA CGA GGA GGT GAT GTC (SEQ. ID. No. 7), based on the sequence containing the signal peptide, and the reverse primer at the C-terminus, AGG ACA AAT TTA AGC ATT GCT GCA TTA TCT ACC (SEQ. ID. No. 8).

Expression of M. pruriens cysteine protease in E. coli—The cDNA (1300 bp) coding for the pro region and the mature portion of the protease was cloned into the expression vector pTWIN1 (New England Biolabs) containing the T7 promoter and lac I repressor. The vector, pTWIN1MUC1300, was transformed into ER2566. The cells growing in log phase were induced with 0.3 mM IPTG. The E. coli were harvested, suspended in 30 ml of lysis buffer (0.5 M NaCl, 0.02 M Tris, 1 mM EDTA, pH8.5) and lysed in a French press. The protein present predominately in the insoluble fraction as inclusion bodies, was solubilized with 8 M urea and refolded in the presence of 0.5 M NaCl, 0.05 M Tris, pH 8.5, 0.7 M L-arginine, 0.01 M glutathione, 0.001 M glutathione disulfide, and 0.005 M EDTA as described in the literature (ref: Hyo-Sung Hwang and Hye-Shin Chung, Protein Expression and Purification, 25, 541-546, 2002). The refolded pro-protease, or zymogen, was activated in the presence of 0.2 M sodium acetate, pH 4.0, 0.005 M DTT and 0.005 M EDTA, and incubated at 37° C. for 30 minutes. The recombinant protein was purified by Centricon Plus-70 concentrators (Millipore) with 30 kDa molecular weight cutoffs and dialyzed extensively against 0.1 M NaCl and 0.01 M L-cysteine, pH 5.7.

Zymogram—The zymogram of native and recombinant mucunain was carried out using a 12.5% native casein-zymogram gel as recommended by the supplier (Biorad). The zymogram gel was run with 0.1 μg of each of the mucunains, renatured and developed for protease activity overnight.

Chromogenic substrate assay—Native and recombinant mucunain protease activity were measured in a buffer containing 50 mM sodium acetate (pH 5.5), 2.5 mM EDTA, 2.5 mM DTT, and 250 μg/ml Z-Phe-Arg-pNA as the chromogenic substrate. After adding protease, the reaction was incubated at room temperature for 1 hr. Generation of the yellow product, p-nitroaniline, was assayed by measuring the absorbance at 405 nm. Papain served as a control cysteine protease. E64, an irreversible inhibitor of cysteine proteases, was used at a concentration of 10 M. Experiments were performed in triplicate and the error bars represent standard deviation.

Reconstitution of spicules—Native spicules were inactivated by autoclaving for 1 hour and reconstituted with native and recombinant mucunains in the presence or absence of cysteine protease inhibitor, E64. Typically, 50 mg of inactivated spicules were mixed with 0.4 ml of 28 μM mucunain in a 2 ml microfuge tube, vortexed, and dried in a Speed-vac for 2 hours at room temperature. These spicules were then considered as ‘reconstituted’ with the protease alone. Spicules prepared in a similar manner but with E64 added at twice the concentration of mucunain, are considered reconstituted with the protease and the inhibitor.

Psychophysics—These protocols were approved by the Yale University Human Investigation Committee. A row of 10 cowhage spicules were lightly pasted to the flattened end of a Weck-Cel surgical strip, spaced at 1 mm, and applied parallel to the surface of the skin at the mid-volar forearm. This resulted in an average of 7 spicules superficially inserted approximately 0.2 mm into skin. Every 30 sec, subjects rated the magnitude of each of three qualities of sensation: Itch, pricking/stinging, and burning using the Generalized Labeled Magnitude Scale (Bartoshuk et al. 2004) that employs appropriately placed intensity descriptors of “none”, “barely detectable”, “weak”, “moderate”, “strong”, “very strong” and, at the top of the scale, “the strongest sensation imaginable.” Voltages derived from each subject's movements of a computer mouse that controlled a cursor on a visual representation of this scale were converted to numbers between 0 and 100 and plotted for each quality at successive intervals of 30 s up to 20 min after the insertion of cowhage. Each of nine subjects received, in separate tests, five types of cowhage spicules: native cowhage spicules, heat-inactivated spicules that had been soaked in a solution of cowhage extract containing E64, or the same but without E64, and heat-inactivated spicules that had been previously soaked in a solution of recombinant mucunain containing E64, or the same but without E64. The subject and experimenter were blinded as to the type of spicule administered.

PAR activation observed by cytoplasmic calcium release—The four human PAR cDNAs were obtained from commercial sources and cloned into the pcDNA3.1 (−) vector (Invitrogen). HeLa cells were transfected with 10 μg of vector cDNA using Lipofectamine 2000 (Invitrogen) and cultured for two days in 96-well glass bottom plates at 30,000 cells/well. Cells were loaded with Fura-2 as follows. The medium was aspirated and replaced with 100 μl of complete DMEM containing 2 μM Fura-2 (Invitrogen) and kept at room temperature in the dark for 1 hr. The medium was again aspirated and each well received 90 μl of HEPES buffered saline (20 mM HEPES, 115 mM NaCl, 5.4 mM KCl, 2 mM CaCl₂, 0.8 mM MgCl₂, 13.8 mM glucose, pH 7.4). Plates were observed using a Zeiss Axiovert 200M microscope equipped with a filter wheel for monitoring excitation at 340 and 380 nm. Axiovision software, version 4.6 was used to analyze ratiometric calcium imaging of the cells. Ten μl of protease was applied approximately 20 seconds after the start of excitation. Images were taken every 5 seconds, beginning at time zero, for 3 minutes. The software was used to analyze the 37 images taken during each 3-minute period.

Results

A series of natural product isolation experiments was undertaken to examine the presence of small molecules and proteins in the spicules scraped or shaken from dried pods collected from Costa Rica, India and Venezuela. Spicules were removed from dry pods via scraping or shaking. Aqueous or organic solvents were used to extract material from spicules. The extract was then subjected to thin layer, size exclusion, anion exchange or reversed phase chromatography. Fractions were then analyzed for chemical components by mass spectrometry and gel electrophoresis. An assumption was made that the active component would likely be relatively abundant. Of nine components identified, two were thus pursued for further study. One was a low molecular weight compound with a mass of 441 Da consistent with folic acid, a known plant component but not a substance implicated in itch. The other was a 36 kDa protein obtained from aqueous extracts (FIG. 1B). The native protein undergoes post-translational modification and thus run more slowly than the recombinant protein under reducing conditions. This protein was subsequently found to evoke itch in human volunteers as described below. Oligonulceotides, designed from the examination of N-terminal and internal amino acid sequences, were used in PCR reactions to obtain the full-length cDNA (FIG. 2) which was then used to express recombinant protein. Analysis of determined and deduced amino acid sequences and nucleotide sequences were consistent with a cysteine protease. The deduced amino acid sequence predicts a 20-residue signal peptide, a pro-region of 70 residues that constitutes the inactive zymogen portion, and a mature protein of 332 amino acids with a predicted mass of 36 kDa. The word ‘mucunain’ was applied to this protein as this term was used in the description of a pruritus-inducing extract from M. pruriens in the 1950's.

A zymogram was run to determine if mucunain had protease activity. Protease activity was found for both native and recombinant mucunain (FIG. 1C). The native and recombinant proteins run similar distances as this is a non-reducing gel. To determine if the activity of mucunain was consistent with cysteine and serine proteases, the capacity for the native and recombinant proteins to cleave Z-FR-pNA, a chromogenic substrate of these proteases was evaluated (FIG. 1D). To distinguish between cysteine and serine protease activity, E64, a small molecule irreversible inhibitor of cysteine proteases (Barrett et al. 1982), was then used in the chromogenic assay. Mucunain cleaves the chromogenic substrate Z-FR-pNA and this cleavage was blocked by the protease inhibitor E64. Papain served as a positive control. The proteases were used at the following concentrations: native mucunain 0.9 μM, recombinant mucunain 2.7 μM and papain 4.3 μM. E64 blocked the protease activity, consistent with mucunain being a cysteine protease as predicted by sequence analysis.

Comparative analysis of active sites of cysteine proteases from other plants with the presumptive active site of the cloned protein (FIG. 2) is consistent with the protein isolated from cowhage being a cysteine protease (Table 2).

The sensory effects of mucunain was further analyzed. A method of introducing mucunain into the skin of volunteers that would mimic the approach associated with the spicules was developed. Such a method would decrease or eliminate the pain associated with using conventional needles as such nociception might interfere with the sensation of itch. Spicules are inactivated when autoclaved but appear to maintain their physical structure. Inactivated spicules were soaked in a solution containing native or recombinant mucunain and then dried. One gram of fresh spicules was stirred for one hour at 4° C. with 20 ml of 0.1 M potassium phosphate buffer (pH 4.8) and 0.9% NaCl and the liquid removed by filtration through cheesecloth. The spicules were teased from the cheesecloth and autoclaved under dry heat for 30 minutes. The mucuna extract was passed through molecular filters to remove components with molecular weights of less than 9 kD. 50 mg of the inactivated, dried spicules were then soaked in 0.05 ml of the filtered extract containing 0.07 mg of cysteine protease (as determined by non-interfering protein assay) with or without 3.5 micrograms of E-64. The spicules were subsequently dried in a Speed-Vac for 2 hours. These spicules were considered ‘reconstituted’ with one batch containing the cysteine protease and its inhibitor and another batch, the protease alone. An analogous approach was used to load spicules with papain or histamine.

The amount of cysteine protease contained in a native spicule was calculated as follows. One gram of dry material contains approximately 700,000 spicules. The aqueous plant extract containing predominantly the 32 kD protein with the properties of a cysteine protease, was assayed for protein content using a Non-Interfering Protein Assay (Geno Technologies, Inc.) and lyophilized. It was found to have a protein content of about 1.4 mg. Assuming that 1.2 of the 1.4 mg of protein is mucunain, in combination with 700,000 spicules per gram, then there are 1.7 ng of mucunain/spicule, which we have rounded up to 2 ng.

Insertion of these reconstituted spicules in the skin of volunteers mimicked the sensations of itch, burning and pricking of the native spicules (FIG. 3, Table 3). Spicules inactivated by autoclaving and then ‘reconstituted’ with mucunain evoked itch and nociceptive sensations consisting of burning and/or pricking/stinging. Addition of E64 during reconstitution produced spicules that did not evoke significant itch or nociceptive sensations. Autoclaved spicules elicited no sensations after insertion into the skin (data not shown). E64 blocked all sensations evoked by spicules containing an extract of native cowhage or recombinant mucunain. Papain, bromelain and histamine all produced itch when applied by this method. Inactivated spicules that were co-incubated with extract, recombinant mucunain or papain did elicit itch. E-64 treated spicules had no effect. Inactivated spicules alone had no effect. These observations suggest that the cysteine protease is the only component of the spicules to cause pruritus. In addition, two short peptide agonists of the PAR4 receptor, GYPGKF-NH₂ (SEQ. ID. No. 28) and AYPGKF-NH₂ (SEQ. ID. No. 30) (Peptides International) were used and incubated with inactivated spicules. They elicited itch (Table 3), at least as well as SLIGRL-NH₂ (SEQ. ID. No. 27), a peptide agonist of the PAR2 receptor known to induce itch in mice. Spicules were incubated with all of these peptides at a concentration of 10 mM. These results are consistent with mucunain being the only itch-inducing component of native spicules as well as accounting for the accompanying sensations of pricking and burning.

The receptor through which mucunain signaled was determined. Other cysteine proteases such as the dust mite cysteine protease der p 1, implicated in allergy, signals through PAR2 (Kauggman et al.) and gingipains, which are cysteine proteases produced by bacteria associated with gingivitis, signal through PAR4 (Afrodite et al.). PAR receptor activation has typically been studied via calcium signaling assays. PAR signaling is complex but is known to include Gq, the activation of which can be followed via calcium mobilization. cDNAs encoding PAR2 and PAR4 were transfected transiently into HeLa cells, loaded with Fura2 and ratiometric calcium imaging obtained following incubation with native mucunain, recombinant mucunain and papain in the absence or presence of E64. Each of the plant proteases was found to activate PAR4, the activation of which was blocked by E64 (FIG. 4). PAR2 was also activated but perhaps to a lesser extent than PAR4. FIG. 5 presents the data collected from FIG. 4 as a histogram. The PAR2 and PAR4 hexapeptide agonists, SLIGRL (SEQ. ID. No. 27) and AYPGKF (SEQ. ID. No. 30) respectively, served as positive controls for the respective receptors. Cysteine proteases mucunain (native and recombinant), papain, bromelain, ficin, and the PAR4 agonist peptide AYPGKF (SEQ. ID. No. 30) all induced calcium influx in PAR4 transfected Hela cells. The cysteine proteases also induced calcium influx in PAR2 transfected Hela cells, although to a much less extend.

The results presented here reveal that the itch-inducing component of the spicules from M. pruriens is mucunain, a cysteine protease, and that itch is mediated primarily via PAR4 receptor. The data also reveal that mucunain and papain activate PAR receptors.

The references cited herein and throughout the specification are incorporated herein by reference.

### REFERENCES

Stander, S., et. al., Clinical classification of itch: a position paper of the international forum for the study of itch. Acta Derm. Venereol. 87:291-294 (2007).
Klein, P. A. & Clark, R. A. F. An evidence-based review of the efficacy of antihistamines in relieving pruritus in atopic dermatitis. Arch. Dermatol. 135: 1522-1525 (1999).
Steinhoff, M., et. al., Proteinase-activated receptor-2 mediates pathway for pruritus in human skin. J. Neurosci. 23: 6176-6180 (2003).
Simone, D. A., et. al., The magnitude and duration of itch produced by intracutaneous injections of histamine. Somatosens. Res. 5:81-92 (1987).
Atanassoff, P. G., et. al., Enhancement of experimental pruritus and mechanically evoked dysesthesias with local anesthesia. Somatosen. Mot. Res. 16: 299-303 (1999).
Johanek. L. M., et. al., Psychophysical and physiological evidence for parallel afferent pathways mediating the sensation of itch J. Neurosci. 27: 7490-7497 (2007).
Masood, K., et. al., Psychophysical measurements of pruritic and nociceptive sensations and dysesthetic states evoked by cutaneous application of cowhage spicules in human. Soc. Neurosci. Abstr. Program No. 50.9 (2005).
Shimada, S. G., et. al., Itch exhibits spatial summation. Neurosci. Abstr. Program No. 552.2/Z28 (2006).
Shelley, W. B. & Arthur, R. P. Studies on cowhage (Mucuna pruriens) and its pruritogenic protease, mucunain. Arch. Dermatol. 72: 399-406 (1955).
Shelley, W. B., Arthur, R. P. Mucunain, the active pruritogenic proteinase of cowhage. Science. 122:469-470. (1955).
Barrett, A. J., et. al., L-trans-Epoxysuccinyl-leucylamido(4-guanidino)butane (E-64) and its analogues as inhibitors of cysteine proteinases including cathepsins B, H and L. Biochem. J. 201: 189-198 (1982).
Shelley, W. B. & Arthur, R. P. The neurohistology and neurophysiology of the itch sensation in man. Arch. Dermatol. 76: 296-323 (1957).
Epidemiologic notes and reports mucuna pruriens-associated pruritus—New Jersey. MMWR Weekly December 06, 34: 732-734 (1985).
Kauggman, H. F., et. al., House dust mite allergens Der p 1 and Der p 5 activate human airway-derived epithelial cells by protease-dependent and protease-independent mechanisms. Clin. Mol. Allergy. 28: 5 (2006).
Jeong, H. H., et. al., German cockroach extract activates protease-activated receptor 2 in human airway epithelial cells. J. All. Clin. Immunol. 113: 315-319 (2004).
Afrodite, L, et. al., Activation of protease-activated receptors by gingipains from Porphyromonas gingivalis leads to platelet aggregation: a new trait in microbial pathogenicity. Blood. 97: 3790-3797 (2001).
Sun, Y-G., & Chen, Z F. A gastrin-releasing peptide receptor mediates the itch sensation in the spinal cord. Nature. 448: 700-703 (2007).

TABLE 1
List of PAR 4 Antagonist

| PAR4 Antgonist name | Chemical Name | Reference |
|---|---|---|
| tcY-NH(₂) | trans-cinnamoyl (Tc)- | Harper MT and Sage SO, |
| | YPGKF-NH(₂) | 2006, Platelet 17: 134-42 |
| | (SEQ. ID. No. 1) | |
| pepducin P4 pal-10 | palmitoyl-SGRRYGHALR- | Houle S et. al. 2005 Br. J |
| pepducin P4 pal-15 | NH(2) (SEQ. ID. No. 25) | Pharmacol. 146: 670-8 |
| Tc-APGKF-NH(₂) | | Hollenberg MD et al. |
| (SEQ. ID. No. 26) | | 2004 Br. J Pharmacol. |
| | | 143: 443-54 |
| Poly-clonal anti-PAR4 | | Kahn M. et al. 1999 J Clin |
| antibody | | Invest 103: 879-887 |
| YD-3 | 1-benzyl-3- | Wu CC et. al 2002 |
| | (ethoxycarbonylphenyl)- | Thromb. Haemost. |
| | indazole | 87: 1026-33 |
| Statin: | | Patent Application |
| Atorvastatin, pravastatin, | | #20050215618 |
| fluvastatin, | | |
| cerivastatin, lovastatin, simva | | |
| statin, rosuvastatin, | | |
| pitavastatin, and metabolite | | |
| thereof | | |
| Ethanol | | Kasuda S. et al. 2006 |
| | | Alcohol Clin Exp. Res. |
| | | 30: 1608-14 |

TABLE 2

Comparison of mucuna cysteine protease active
site sequence to such sites in other cysteine
proteases.

| | |
|---|---|
| Cysteine protease | |
| source | Cysteine active site motif |

| | | |
|---|---|---|
| Mucuna (mucunain) | QGSCGSCWAFS | (SEQ. ID. No. 9) |
| Papaya (papain) | QGSCGSCWAFS | (SEQ. ID. No. 10) |
| Pineapple | QNPCGSCWAFS | (SEQ. ID. No. 11) |
| (bromelain) | | |
| Fig (ficin) | QGQCGSCW--- | (SEQ. ID. No. 12) |
| Arabidopsis | QGSCGSCWAFS | (SEQ. ID. No. 13) |
| Pea | QGSCGSCWAFS | (SEQ. ID. No. 14) |
| Rice | QGQCGCCWAFS | (SEQ. ID. No. 15) |
| Broccoli | QGSCGSCWAFS | (SEQ. ID. No. 16) |
| Kidney bean | QGSCGSCWAFG | (SEQ. ID. No. 17) |
| Kiwi fruit | QGECGGCWAFS | (SEQ. ID. No. 18) |
| (actinidin) | | |
| Ginger | QGRCGSCWAFA | (SEQ. ID. No. 19) |
| Cathepsin B (human) | QGSCGSCWAFG | (SEQ. ID. No. 20) |
| Cathepsin S (human) | QGSCGACWAFS | (SEQ. ID. No. 21) |
| Dust mite (Der p 1) | QGGCGSCWAFS | (SEQ. ID. No. 22) |
| Caspase-1 (human) | KPKVIIIQACRG | (SEQ. ID. No. 23) |
| (serine protease) | | |
| Caspse-3 (human) | KPKLFIIQACRG | (SEQ. ID. No. 24) |
| (serine protease) | | |

TABLE 3
Activity of reconstituted spicules and the effects of E-64

| Status of spicules | Itch sensation |
|---|---|
| Fresh spicules | + |
| Fresh spicules + E-64 | − |
| Inactive spicules | − |
| Inactive spicules + Mucuna extract | + |
| Inactive spicules + Mucuna extract + E-64 | − |
| Inactive spicules + recombinant mucunain | + |
| Inactive Spicules + recombinant mucunain + E-64 | − |
| Inactive spicules + Papain | + |
| Inactive spicules + papain + E-64 | − |
| Inactive spicules + Bromelain | + |
| Inactive spicules + Bromelain + E-64 | − |
| Inactive spicules + SLIGRL (PAR2 agonist) | + |
| (SEQ. ID. No. 27) | |
| Inactive spicules + GYPGKF (PAR4 agonist) | + |
| (SEQ. ID. No. 28) | |

We claim:

1. A method of preventing and/or treating itch in a subject in need thereof comprising administering a therapeutically effective amount of a PAR4 antagonist to the subject.

2. The method of claim 1, wherein said PAR 4 antagonist is selected from the group consisting of tcY-NH(₂); pepducin P4 pal-10; pepducin P4 pal-15; Tc-APGKF-NH(₂) (SEQ. ID. No. 26); polyclonal anti-PAR4 antibody; monoclonal anti-PAR4 antibody, YD-3; Statins: atorvastatin, pravastatin, fluvastatin, cerivastatin, lovastatin, simvastatin, rosuvastatin, pitavastatin, and metabolite thereof; and ethanol.

3. The method of claims 1-2, wherein the itch is associated with a disease or disorder selected from eczema, atopic eczematous dermatitis, seborrheic dermatitis, atopic dermatitis, contact dermatitis, irritant dermatitis, xerosis (dry skin), psoriasis, fungal infections including athlete's foot, yeast infections including diaper rash and vaginal itch, parasitic infections, parasitic infestations including scabies and lice, lichen planus, lichen simplex, lichen simplex chronicus, lichen sclerosis, itch secondary to medications, senile itch, uremia, idiopathic itch, itch associated with liver cirrhosis, itch associated with inflammation, itch associated with allergies, itch associated with cancer, itch associated with kidney disease, itch associated with haemodialysis, burns, scalds, sunburn, wound healing, insect bites, urticaria, sweat gland abnormalities, bullous pemphigoid, photodematoses, skin blisters, adult acne, chicken pox, and dermatitis herpetiformis.

4. The method of claims 1-3, wherein said PAR4 antagonist is administered in the form of a pharmaceutical composition comprising a therapeutically effective amount of said PAR4 antagonist as active ingredient, in admixture with a pharmaceutical carrier.

5. The method of claims 1-4, wherein said PAR4 antagonist is applied topically to the site afflicted with itch.

6. The method of claim 5, wherein said pharmaceutical composition is formulated in a form suitable for topical application.

7. The method of claim 6, wherein said pharmaceutical composition comprising reconstituted spicules from the pods of the tropical plant Mucuna pruriens.

8. The method of claims 1-7, wherein said PAR4 antagonist is administered systemically.

9. The method of claims 1-9, further comprising administering a therapeutic amount of a PAR2 antagonist.

10. A method of preventing and/or treating itch in a subject in need thereof comprising administering a therapeutically effective amount of the cysteine protease inhibitor, E-64, to the subject.


SEQUENCE LISTING (33 sequences)

SEQ ID NO: 1 - protein, 5 aa, Artificial Sequence
  Description of Artificial Sequence Synthetic peptide
YPGKF

SEQ ID NO: 2 - DNA, 30 bp, Artificial Sequence
  Description of Artificial Sequence Synthetic primer
tggggcgccagctggggyttcgagggytay

SEQ ID NO: 3 - protein, 14 aa, Artificial Sequence
  Description of Artificial Sequence Synthetic peptide
NSWGASWGFEGYVR

SEQ ID NO: 4 - DNA, 33 bp, Artificial Sequence
  Description of Artificial Sequence Synthetic primer
aggacaaatttaagcattgctgcattatctacc

SEQ ID NO: 5 - DNA, 29 bp, Artificial Sequence
  Description of Artificial Sequence Synthetic primer
gataacttgccggaatctgttgattggag

SEQ ID NO: 6 - protein, 20 aa, Artificial Sequence
  Description of Artificial Sequence Synthetic peptide
DNLPESVDWRNEGAVLPCKS

SEQ ID NO: 7 - DNA, 30 bp, Artificial Sequence
  Description of Artificial Sequence Synthetic primer
cacgtggcggagcgacgaggaggtgatgtc

SEQ ID NO: 8 - DNA, 33 bp, Artificial Sequence; identical to SEQ ID NO: 4
  Description of Artificial Sequence Synthetic primer

SEQ ID NO: 9 - protein, 11 aa, Mucuna pruriens
QGSCGSCWAFS

SEQ ID NO: 10 - protein, 11 aa, Carica papaya
QGSCGSCWAFS

SEQ ID NO: 11 - protein, 11 aa, Ananas comosus
QNPCGSCWAFS

SEQ ID NO: 12 - protein, 8 aa, Ficus sp.
QGQCGSCW

SEQ ID NO: 13 - protein, 11 aa, Arabidopsis sp.
QGSCGSCWAFS

SEQ ID NO: 14 - protein, 11 aa, Pisum sativum
QGSCGSCWAFS

SEQ ID NO: 15 - protein, 11 aa, Oryza sativa
QGQCGCCWAFS

SEQ ID NO: 16 - protein, 11 aa, Brassica sp.
QGSCGSCWAFS

SEQ ID NO: 17 - protein, 11 aa, Phaseolus vulgaris
QGSCGSCWAFG

SEQ ID NO: 18 - protein, 11 aa, Actinidia deliciosa
QGECGGCWAFS

SEQ ID NO: 19 - protein, 11 aa, Zingiber officinale
QGRCGSCWAFA

SEQ ID NO: 20 - protein, 11 aa, Homo sapiens
QGSCGSCWAFG

SEQ ID NO: 21 - protein, 11 aa, Homo sapiens
QGSCGACWAFS

SEQ ID NO: 22 - protein, 11 aa, Dermatophagoides sp.
QGGCGSCWAFS

SEQ ID NO: 23 - protein, 12 aa, Homo sapiens
KPKVIIIQACRG

SEQ ID NO: 24 - protein, 12 aa, Homo sapiens
KPKLFIIQACRG

SEQ ID NO: 25 - protein, 10 aa, Artificial Sequence
  Description of Artificial Sequence Synthetic peptide
SGRRYGHALR

SEQ ID NO: 26 - protein, 5 aa, Artificial Sequence
  Description of Artificial Sequence Synthetic peptide
APGKF

SEQ ID NO: 27 - protein, 6 aa, Artificial Sequence
  Description of Artificial Sequence Synthetic peptide
SLIGRL

SEQ ID NO: 28 - protein, 6 aa, Artificial Sequence
  Description of Artificial Sequence Synthetic peptide
GYPGKF

SEQ ID NO: 29 - protein, 6 aa, Artificial Sequence
  Description of Artificial Sequence Synthetic peptide
GYPGKF

SEQ ID NO: 30 - protein, 6 aa, Artificial Sequence
  Description of Artificial Sequence Synthetic peptide
AYPGKF

SEQ ID NO: 31 - protein, 6 aa, Artificial Sequence
  Description of Artificial Sequence Synthetic peptide
FSLLRY

SEQ ID NO: 32 - DNA, 1585 bp, Artificial Sequence
  Description of Artificial Sequence Synthetic construct
cacgtggcggagcgacgaggaggtgatgtcgttgtacgaacagtggctggtgaagcatggaaaggcgtacaacgctctcggggagaaggacaagaggttcgacatcttcaaagacaacctgcgcttcatcgacgaccacaacgccgacaaccgaacctacaagctcggtctcaaccgcttcgcggatctcaccaacgaagagtacagagccaggtacttgggaaccaggatcgatccgaaccggaggttcgttaagaccaagacccagagcaaccgttacgcgccacgtgtcggggacaatctaccggaatccgttgattggagaaacgagagtgctgttctcccagtcaaagaccaaggaaactgtgggagctgttgggcattctcaacaattggtgcggtggaaggaataaacaagatagtaacaggcgatctaatttcattatcagaacaagagttagtggattgtgatacatcatataatcaaggatgcaatggaggactcatggactatgcctatgaattcatcatcaacaatggaggcattgattccgaggaggattacccttaccgtgccgttgatggtacttgtgaccaatataggaaaaatgctaaagttgttaccattgattcctacgaagatgttcctgccaacgacgagctagccttgaaaaaggctgttgcaaatcagcctgtgagcgtagctattgaaggaggaggcagggaatttcaattatatgtttctggtgtatttactgggagatgtgggacagcactagatcatggtgttgtggctgttgggtatggttcagttaaaggtcatgattattggattgtaaggaattcatggggtgctagctggggagaggagggctatgtgagattagagagaaatctggctaagagcagatcaggaaagtgtggaattgcaattgagccatcttatcccatcaagaatggtgcaaatccccccaatcctggaccatcacctccttcaccagtgaagccaccaaatgtttgtgacaactcctacagctgctctgatagtgctacttgttgctgtattttcgagtttcaaaaatattgcatggtgtggggttgctgtcctctagaggctgctacctgctgtgacgaccattacagttgctgccctcatgaatatcccatctgcaacgttcgtgctggaacttgtttgaagggcaagaacaacccatttggagtgaaggcattgaggcgtactccagctaaaccccattgggcctttggaggtaagaacaaggtgaacagtgcttaagaagttaaagggaacgtgtacgtgcagaaggagtcgatgaaggactaaaaacactagctgagctgatacacttcacgtaatcctgtacgtagcatgcagatcttacagccaatttattatgatacaaacatattctgtatcaaatttcgtcatacattggctctgtacataggattatattaattatgcactgtatttgtattcaaattccaattgccgaacatatttcattgctgttattttccaacttcggtagataatgcagcaatgcttaaatttgtcctttattca

SEQ ID NO: 33 - protein, 422 aa, Artificial Sequence
  Description of Artificial Sequence Synthetic construct
MSLYEQWLVKHGKAYNALGEKDKRFDIFKDNLRFIDDHNADNRTYKLGLNRFADLTNEEYRARYLGTRIDPNRRFVKTKTQSNRYAPRVGDNLPESVDWRNESAVLPVKDQGNCGSCWAFSTIGAVEGINKIVTGDLISLSEQELVDCDTSYNQGCNGGLMDYAYEFIINNGGIDSEEDYPYRAVDGTCDQYRKNAKVVTIDSYEDVPANDELALKKAVANQPVSVAIEGGGREFQLYVSGVFTGRCGTALDHGVVAVGYGSVKGHDYWIVRNSWGASWGEEGYVRLERNLAKSRSGKCGIAIEPSYPIKNGANPPNPGPSPPSPVKPPNVCDNSYSCSDSATCCCIFEFQKYCMVWGCCPLEAATCCDDHYSCCPHEYPICNVRAGTCLKGKNNPFGVKALRRTPAKPHWAFGGKNKVNSA